(19) United States
(12) Patent Application Publication
(10) Pub. No.: US 2011/0004224 A1
(43) Pub. Date: Jan. 6, 2011

(54) TRACKING CAS SYSTEM

(76) Inventors: Emmanuel Daigneault, Montreal (CA); Ahmed Aoude, Pointe-Claire (CA)

Correspondence Address: Ogilvy Renault LLP (Zimmer Cas), 1 Place Ville Marie, Suite 2500, Montreal, QC, H3B 1R1, CA

(21) Appl. No.: 12/679,493

(22) PCT Filed: Mar. 12, 2009
(86) PCT No.: PCT/CA09/00317

§ 371 Date: Apr. 1, 2010

Related U.S. Application Data
(60) Provisional application No. 61/036,204, filed on Mar. 13, 2008.

(51) Int. Cl. A61B 19/00

(52) U.S. Cl. 606/130

(57) ABSTRACT

A computer-assisted surgery system for tracking an object during surgery comprises a first tracker device and a second tracker device secured to different parts of the object. The first and the second tracker device are secured to the object separately in such a way that a geometrical pattern is defined with trackable elements from the first tracker device and from the second tracker device. A tracking system has a sensor unit tracking the trackable members. A calibration unit defines a geometrical pattern from the trackable members. A pattern identifier identifies the geometrical pattern. A position and orientation calculator calculates a position and orientation of the geometrical pattern The tracking system calculates position and orientation of the object as a function of the position and orientation of the geometrical pattern and of object/geometrical pattern relation data gathered during surgery.

### CROSS-REFERENCE TO RELATED APPLICATION

This patent application claims priority on U.S. Provisional Application No. 61/036,204, filed on Mar. 13, 2008.

### FIELD OF THE APPLICATION

The present application relates to computer-assisted surgery systems and, more particularly, to instrumentation used for the tracking of surgical tools during computer-assisted surgery.

### BACKGROUND OF THE ART

Tracking of surgical instruments or tools is an integral part of computer-assisted surgery (hereinafter CAS). The tools are tracked for position and/or orientation in such a way that information pertaining to bodily parts is obtained. The information is then used in various interventions with respect to the body, such as bone alterations, implant positioning, incisions and the like.

Two types of tracking system are commonly used. The active tracking systems provide a transmitter on the tool to be tracked, which transmitter emits signals to be received by a processor of the CAS system, which will calculate the position and/or orientation of the tool as a function of the signals received. The transmitters of the active tracking systems are powered, for instance by being wired to the CAS system or by being provided with an independent power source, so as to emit signals.

Passive tracking systems do not provide active transmitters on the tools, and therefore represent fewer issues pertaining to sterilization. The CAS system associated with passive tracking has an optical sensor apparatus provided to visually detect optical elements on the tools. The optical elements are passive, whereby no power source is associated therewith.

In order to obtain values for position and/or orientation, the optical elements must be in the line of sight of the optical sensor apparatus. Accordingly, with passive tracking systems, surgery takes place in a given orientation as a function of the required visibility between the optical sensor apparatus and the optical elements.

It is desirable to reduce the invasiveness of surgery for numerous reasons. For instance, invasive surgery leaves greater scars on the soft tissue of the patient. Also, the use of numerous tracking devices bolted directly to the bone elements tends to weaken the bone elements, potentially increasing the risks of post-surgical fractures. Finally, bigger incisions in the soft tissue and multiple holes in the bone elements tend to increase the pain of the patient in the post-surgical period.

### SUMMARY OF THE APPLICATION

It is therefore an aim of the present application to provide a novel tracking CAS system and method.

Therefore, in accordance with a first embodiment, there is provided computer-assisted surgery system for tracking an object during surgery, comprising: a first tracker device secured to a first part of the object, the first tracker device having a first trackable member; a second tracker device secured to a second part of the object, the second tracker device having at least a second and a third trackable member, the first and the second tracker device being secured to the object separately in such a way that a geometrical pattern is defined with trackable elements from the first tracker device and from the second tracker device; a tracking system having a sensor unit tracking the trackable members, a calibration unit for defining a geometrical pattern from the at least three trackable members, a pattern identifier for identifying the geometrical pattern, and a position and orientation calculator for calculating a position and orientation of the geometrical pattern; the tracking system calculating a position and orientation of the object as a function of the position and orientation of the geometrical pattern and of object/geometrical pattern relation data gathered during surgery.

Further in accordance with the first embodiment, the first tracker device is anchored directly to the bone.

Still further in accordance with the first embodiment, the second tracker device is immovably secured to soft tissue.

Still further in accordance with the first embodiment, the second tracker device is a strap immovably secured to the soft tissue, the strap having patches of retroreflective material detectable by the sensor unit to define the at least second and third trackable members.

Still further in accordance with the first embodiment, the first tracker device and the second tracker device are secured to opposite ends of a same bone.

In accordance with a second embodiment, there is provided a method for defining a frame of reference for a bone element in computer-assisted surgery, comprising: securing tracker devices each having at least one trackable member at two different locations on the bone element; aligning the trackable members of both said tracker devices with a sensor apparatus; and initiating a calibration whereby a geometrical pattern is defined with the trackable members of both tracker devices; whereby the geometrical pattern is subsequently tracked to define a frame of reference for the bone element.

Further in accordance with the second embodiment, securing the tracker devices comprises securing a first of the tracker devices directly to a bone element.

Still further in accordance with the second embodiment, securing the tracker devices comprises immovably securing a second of the tracker devices to the bone element on soft tissue.

Still further in accordance with the second embodiment, securing the tracker devices comprises securing the tracker devices to opposite ends of the same bone element.

Still further in accordance with the second embodiment, the method is performed on a cadaver or a model.

### BRIEF DESCRIPTION OF THE DRAWINGS

FIG. 1 is a schematic view of a leg having tracker devices and tracker straps of the optical tracking CAS system of the present application; and

FIG. 2 is a block diagram of the tracking CAS system of the present application.

### DESCRIPTION OF THE PREFERRED EMBODIMENTS

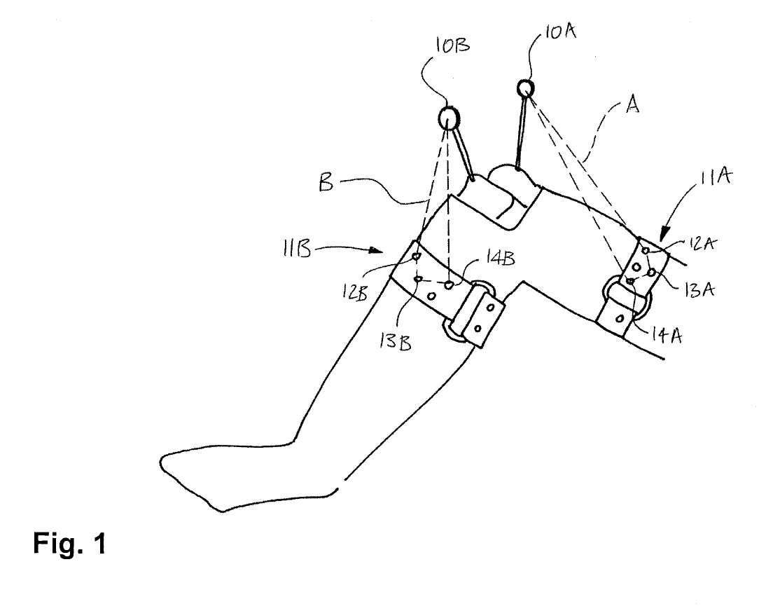

Referring to the drawings and more particularly to FIG. 1, a leg is illustrated with the soft tissue separated to expose the tibial bone and a femoral bone.

In order for an object to be tracked in space for position and orientation, at least two points associated with the object must be known. With two points, the object can be tracked for position and orientation under specific conditions (e.g., object and the two tracked points being collinear, and no view interruption after calibration). A geometrical pattern of three nonlinear trackable points is commonly used for six-degree-of-freedom tracking, and more trackable points can be used for increased precision in the tracking.

The femoral bone is illustrated as supporting a tracker device 10A, whereas the tibial bone supports a tracker device 10B. More specifically, as is commonly known, the tracker devices 10A and 10B are secured directly to their respective bones, for instance using screws. The tracker devices 10A and 10B each have a trackable member thereon. The trackable members typically are tokens or spheres of retroreflective material having a given geometry. Accordingly, the tracker devices 10A and 10B illustrated in FIG. 1 each represent one optically trackable point. However, other types of tracker devices incorporating multiple points can be used as well (e.g., flat geometrical patterns using Scotch-Lite™ patches).

Tracker straps 11A and 11B are respectively secured to the thigh and to the shin, on the soft tissue. The tracker straps 11 are made of a flexible material that is rigidly secured to the soft tissue, in such a way that movement between the straps 11 and their respective bones (femoral bone and tibial bone in FIG. 1) is negligible. For instance, it is considered to provide the straps 11 with a Velcro™ closure system, so as to manually secure the straps 11 about the leg or like member as is illustrated in FIG. 1. It is pointed out that no letter is affixed to the strap 11, to refer to both straps 11A and 11B. This nomenclature is used throughout the present description.

Each strap 11 features a plurality of trackable members, namely patches of retroreflective material forming a cloud of distinct geometrical shapes that are optically detectable. In FIG. 1, some of these trackable members are identified as 12, 13 and 14.

Accordingly, geometrical patterns are defined with a combination of trackable members 12-14 on the straps 11, and optionally with the tracker device 10, such that a sensor apparatus of a CAS system visually recognizes the given geometry, in the case of the illustrated embodiment. More specifically, geometrical pattern A is the frame of reference for the femoral bone, whereas geometrical pattern B is the frame of reference for the tibial bone. The geometrical pattern A consists of the trackable element of the tracker device 10A, and the trackable elements 12A, 13A and 14A of the strap 11A. In this case, the tracker device 10A may be used to confirm that the strap 11A has not moved on the limb of the patient, as the strap 11A is secured to soft tissue. The geometrical pattern B consists of the trackable element of the tracker device 10B, and the trackable elements 12B, 13B and 14B of the strap 11B, again with the tracker device 10B being used to confirm that the strap 11B has not moved on the limb.

It is pointed out that more than one geometrical pattern may be used for a limb. For instance, as described above, the trackable members 12A-14A on the strap 11A may define a first geometrical pattern for the femur, and the tracker device 10A and the trackable members 12A and 13A may define a second geometrical pattern for the femur, such that the tracking system tracks the femur by dynamically switching from one geometrical pattern to the other.

With the tracking of the geometrical patterns A and B, the CAS system calculates a position and/or orientation of the tracked element (i.e., femoral bone or tibial bone) associated with the tracker devices 10 and tracker straps 11. Therefore, the CAS system calculates the position and orientation of the tracked element from the optical tracking of either one of the triangular geometries A and B. As the tracking is optical in the illustrated embodiment, there must be a line of sight between the optical sensor apparatus and the points forming the geometrical patterns A and B. The multiple trackable members (e.g., 12-14) on the straps 11 offer different combinations of points to form geometrical patterns that are in the line of sight of the optical sensor apparatus.

Although three non-linear points in a scalene triangle form a suitable geometrical pattern for optical tracking purposes, the use of four or more points can increase the precision of the optical tracking. Accordingly, the geometrical patterns A and B of FIG. 1 are made of four points. It is also considered to track pentagonal, hexagonal, and other polygonal geometrical patterns.

As mentioned above, different types of retro-reflective trackable members can be used for the tracker devices 10 and the straps 11. For instance, the retro-reflective patches on the straps 11 are made of a material like Scotch-Lite™, whereas the trackable members of the devices 10 are retroreflective spheres.

It is pointed out that FIG. 1 features two independent geometrical patterns, namely patterns A and B, for illustrative purposes, but the leg could be provided with a single combination of tracker device and tracker strap, either for the femoral bone or for the tibial bone, or any other bone element or bodily part that is to be tracked. For instance, only one of the patterns A and B is required in some types of surgeries (e.g., given types of total knee replacement)

The strap 11 constitutes one solution for a non-rigid tracker device that can be temporarily secured to soft tissue in such a way that there is negligible displacement between the tracker device and the bone element. Other configurations are considered, using flexible material supporting retroreflective trackable patterns, or trackable bases snap-fitted or glued to the flexible material, amongst numerous possibilities. Moreover, although the illustrated embodiment uses optical tracking, other types of tracking are considered as well, such as a magnetic tracking, RF tracking, ultrasound tracking.

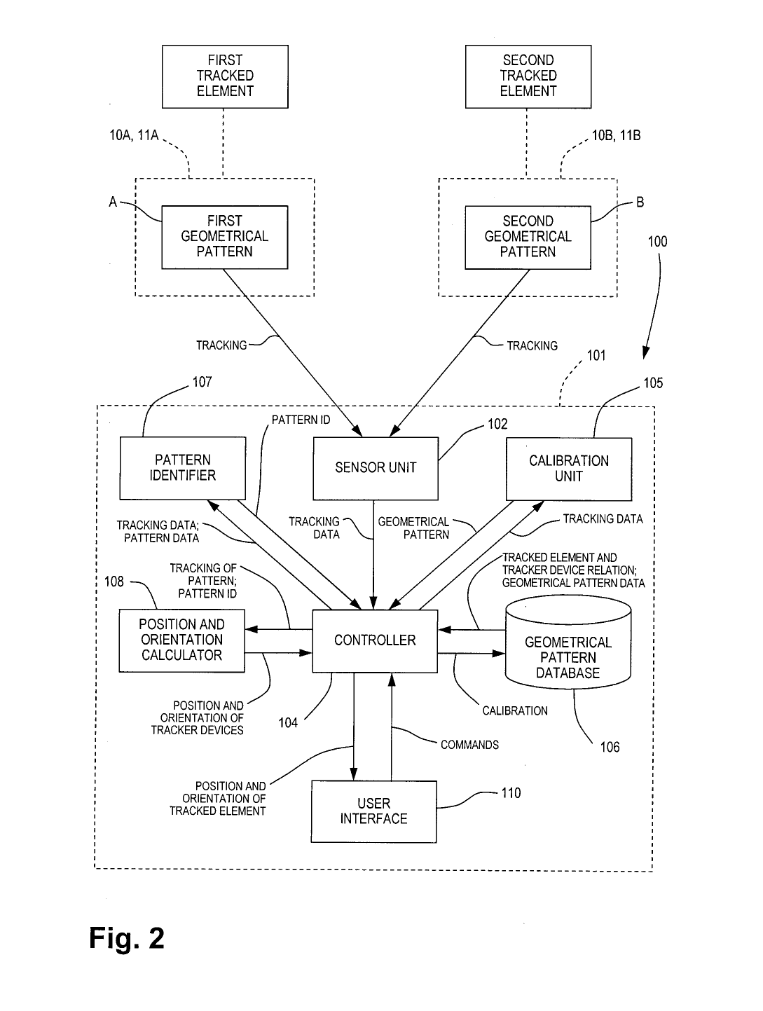

Referring to FIG. 2, a tracking computer-assisted surgery system using the tracker devices 10A and 10B as well as the tracker straps 11A and 11B is generally illustrated at 100. The computer-assisted surgery system 100 incorporates the tracker devices 10A and 10B, and the tracker straps 11A and 11B, as described above.

In accordance with FIG. 1, the combination of the tracker device 10A and the tracker strap 11A, and the combination of the tracker device 10B and the tracker strap 11B, each provide at least one detectable geometrical pattern (A and B, respectively in FIG. 1). The recognition of the geometrical patterns A and B may result from a calibration performed in the first steps of use of the computer-assisted surgery system.

The computer-assisted surgery system has a tracking system 101, which is typically a computer having a processor. The tracking system 101 has a sensor unit (i.e., optical sensor apparatus) provided in order to visually track the tracker devices 10A and 10B, as well as trackable members 12-14 and others of the tracker straps 11. Typically, the sensor unit 102 involves a pair of sensors that are part of a computer-assisted surgical navigation system (e.g., Navitrack™).

A controller 104 is connected to the sensor unit 102. Therefore, the controller 104 receives the tracking data from the sensor unit 102.

A calibration unit 105 is connected to the controller 104. The calibration unit 104 is used to define geometrical patterns, such as A and B in FIG. 1, which will be used throughout surgery to track the object (i.e., bone element). Considering that the at least three points that constitute the geometrical patterns reach a fixed position with respect to the object during surgery, as opposed to preoperatively, the calibration unit 105 defines the geometrical patterns when required. The computer-assisted surgery system prompts an operator to define geometrical patterns, or the operator requests that the geometrical patterns be defined. As mentioned previously, more than one pattern may be defined for a same limb, for instance to increase the range of tracking of the limb. The computer-assisted surgery system dynamically switches from one pattern to another to track the limb.

A database 106 is provided so as to store the geometrical pattern data. More specifically, the various geometrical patterns A and B, which have been defined by the calibration unit 105, are stored in the database 106. Similarly, the relation between the tracked elements (e.g., femoral bone and tibial bone) and the geometrical patterns A and B is stored in the database 106. The tracked element/geometrical pattern relation is subsequent to the calibration performed in the first steps of use of the computer-assisted surgery system, and typically results from operations related to the surgical procedure, such as the digitization of points using a registration pointer.

A pattern identifier 107 is associated with the controller 104. The pattern identifier 107 receives the tracking data from the sensor unit 102 and the geometrical pattern data from the calibration unit 105 or from the database 106, so as to identify which one of the geometrical patterns A and/or B is being tracked. If multiple patterns are visible or tracked, it is preferred that the pattern having the greatest distance between its optical elements or the most points (e.g., quadrilateral over triangles) be selected to reduce the error.

A position and orientation calculator 108 is associated with the controller 104. The position and orientation calculator 108 receives the identification of patterns from the controller 104, which identification of patterns results from the pattern identification by the pattern identifier 107 and from the tracking data. With the identification of the patterns being tracked, the position and orientation calculator 108 calculates the position and orientation of the geometrical patterns A and/or B.

The position and orientation information of the geometrical patterns A and/or B is sent to the controller 104. The controller 104 will combine this information with the tracked element/geometrical pattern relation from the geometrical pattern database 106, so as to calculate the position and orientation of the tracked elements (e.g., femoral bone and tibial bone).

This information is sent to the user interface 110 or to other components of the CAS system for further processing, such that the user of the computer-assisted surgery system obtains information pertaining to the position and orientation of the tracked element in the various forms known to computer-assisted surgery (e.g., visual representation, numerical values such as angles, distances, etc.). It is pointed out that the database 106 may, as well, be part of the controller 104, the pattern identifier 107 or the position and orientation calculator 108.

In order to use the combination of tracker device 10 and strap 11, the following sequence of steps is performed, in any suitable order, or anchoring the tracker device 10 in any suitable fashion such that the tracker device 10 is fixed to the bone.

The tracker device 10 is secured to the bone element that is to be tracked during the surgical procedure. This step typically involves exposing the bone element and screwing the tracker device 10 thereto.

The strap 11 is secured to the soft tissue about the bone element. Both the tracker device 10 and the strap 11 must be in the line of sight of the sensor apparatus of the CAS system.

A calibration is performed so as to define at least one geometrical pattern consisting of points from the tracker device 10 and from the trackable members on the strap 11. A plurality of geometrical patterns can be defined for the same combination of tracker device 10 and strap 11, for instance to increase a range of visibility of the combination.

Once the calibration has been performed and geometrical patterns have been defined, the steps associated with the computer-assisted surgical procedure may be performed. These steps depend on the type of surgery, and includes the resection or alterations of bones using tracked tools, the digitization of surfaces using a registration pointer, defining frames of reference for the bone elements, and other steps involving the CAS system.

Throughout surgery, the tracker device 10 and the trackable members on the strap 11 are then tracked, for the CAS system 100 to identify the geometrical patterns.

Once the geometrical pattern is identified, the position and orientation of the geometrical pattern is calculated. With the position and orientation of the geometrical pattern, data gathered during the computer-assisted surgical procedure is related to the bone elements, using the bone element/geometrical pattern relation data.

1. A computer-assisted surgery system for tracking an object during surgery, comprising:
a first tracker device secured to a first part of the object, the first tracker device having a first trackable member;
a second tracker device secured to a second part of the object, the second tracker device having at least a second and a third trackable member, the first and the second tracker device being secured to the object separately in such a way that a geometrical pattern is defined with trackable elements from the first tracker device and from the second tracker device;
a tracking system having:
a sensor unit tracking the trackable members;
a calibration unit for defining a geometrical pattern from the at least three trackable members;
a pattern identifier for identifying the geometrical pattern; and
a position and orientation calculator for calculating a position and orientation of the geometrical pattern;
the tracking system calculating a position and orientation of the object as a function of the position and orientation of the geometrical pattern and of object/geometrical pattern relation data gathered during surgery.

2. The computer-assisted surgery system according to claim 1, wherein the first tracker device is anchored directly to the bone.

3. The computer-assisted surgery system according to claim 2, wherein the second tracker device is immovably secured to soft tissue.

4. The computer-assisted surgery system according to claim 1, wherein the second tracker device is immovably secured to soft tissue.

5. The computer-assisted surgery system according to claim 4, wherein the second tracker device is a strap immovably secured to the soft tissue, the strap having patches of retroreflective material detectable by the sensor unit to define the at least second and third trackable members.

6. The computer-assisted surgery system according to claim 1, wherein the first tracker device and the second tracker device are secured to opposite ends of a same bone.

7. A method for defining a frame of reference for a bone element in computer-assisted surgery, comprising:
securing tracker devices each having at least one trackable member at two different locations on the bone element;
aligning the trackable members of both said tracker devices with a sensor apparatus; and
initiating a calibration whereby a geometrical pattern is defined with the trackable members of both tracker devices;
whereby the geometrical pattern is subsequently tracked to define a frame of reference for the bone element.

8. The method according to claim 7, wherein securing the tracker devices comprises securing a first of the tracker devices directly to a bone element.

9. The method according to claim 8, wherein securing the tracker devices comprises immovably securing a second of the tracker devices to the bone element on soft tissue.

10. The method according to claim 7, wherein securing the tracker devices comprises immovably securing one of the tracker devices to the bone element on soft tissue.

11. The method according to claim 7, wherein securing the tracker devices comprises securing the tracker devices to opposite ends of the same bone element.

12. The method according to claim 7, wherein the method is performed on a cadaver or a model.
